(19) United States
(12) Patent Application Publication
(10) Pub. No.: US 2017/0196645 A1
(43) Pub. Date: Jul. 13, 2017

(54) PROTECTIVE COVER COATING DEVICE

(71) Applicant: Sükrü Fatih ÖZKARSLI, Istanbul (TR)

(72) Inventor: Sükrü Fatih ÖZKARSLI, Istanbul (TR)

(21) Appl. No.: 15/316,630

(22) PCT Filed: Jun. 4, 2015
(86) PCT No.: PCT/TR2015/000250

§ 371 Date: Dec. 6, 2016

(30) Foreign Application Priority Data
Jun. 6, 2014 (TR) 2014/06485

(51) Int. Cl. A61B 46/10; A61B 42/50; A61C 1/16; B29C 63/20; A61B 8/12; A61M 37/00; B65B 53/00; A61B 1/00; A61B 1/313

(52) U.S. Cl. CPC A61B 46/10; A61B 1/00142; A61B 42/50; A61C 1/16; A61B 1/3132; A61B 8/12; A61M 37/0076; B65B 53/00; B29C 63/20; B29L 2031/753

(57) ABSTRACT

A coating device is provided which allows for coating the surface of devices and tools in direct/indirect contact with the patient in medical and dental areas. Also provided are a coating method that is performed with the coating device and a method for removing the protective cover from the coated devices.

### TECHNICAL FIELD

The invention is related to a coating device that is used for coating devices and tools utilized in medical and dental areas and to the coating method that is performed using said coating device.

The invention is particularly related to a coating device which allows for coating the surface of devices and tools that are in direct or indirect contact with the patient in medical and dental areas with a latex or similar flexible protective cover which may or may not be sterile and to the coating/removing method that is performed with said coating device.

### STATE OF THE ART

Today, fast spreading of viral and communicable diseases threatens the assisting staff and the patients as well as the doctors in the medical and dental working environments. Besides, similar cases are experienced in the tools used for tattooing.

Especially, surfaces of the devices and tools used for treatment in dental offices are contaminated with blood, saliva, debris and other microbial wastes, and it leads to cross-transmission of pathogenic microorganisms from patient to doctor, patient to assisting staff or patient to patient.

In doctors' offices, it is recommended to use autoclave in order to sterilize those devices and tools which are in direct contact with the oral cavity and suitable for autoclave sterilization. It is required to clean the devices thoroughly before entering the autoclave. However, standard autoclaves employed only provide sterilization and the cleaning process is performed manually. And, manual cleaning is a long and difficult process. Besides, the assisting staffs attention and sensitivity is trusted with this cleaning process which is quite important.

Devices such as dental high speed handpiece, electric micromotor, surgical handpiece, ultrasonic scaler, light curing device, and intra-oral camera which are in direct contact with the oral cavity and cannot always be sterilized completely in the autoclave due to their complex designs and electronic components. In order to sterilize said devices in the autoclave after each patient and to re-use them, the number of each device available in the doctor's office should be at least 50-100 which is almost impossible because of the high costs of these devices.

Due to the abovementioned disadvantages, some solutions are proposed to protect dental and medical devices with a protective cover. One of the patents with respect to the subject matter in the literature is the U.S. Pat. No. 8,235,720B1. Said application relates to a cover used to provide hygiene for a dental device. The cover of the invention comprises some components also to contribute to the dental operation.

The patent No. FR2908037 is related to a protection assembly that is used to provide hygiene for syringes especially utilized in a dental environment.

Some U.S. patent applications with respect to the subject matter are the applications numbered as follows: U.S. Pat. No. 1,485,963, U.S. Pat. No. 2,073,137, U.S. Pat. No. 4,728,290, U.S. Pat. No. 4,789,336, U.S. Pat. No. 5,228,851, and U.S. Pat. No. 6,350,124. However, the protective covers proposed for dental handpieces in these applications are produced specifically for each device. The covers designed individually for each device restrict the area of use of the covers. Moreover, it is required to coat said protective covers by manual manipulation. Then again, a secondary manual process is needed while creating drill connection holes for the device. This prolongs the process.

In conclusion, an improvement is deemed necessary in the respective technical field due to the abovementioned drawbacks and inadequacy of the existing solutions with respect to the subject.

### OBJECTS OF THE INVENTION

The present invention relates to a protective cover coating device for medical devices and to the method thereof meeting the abovementioned requirements, eliminating all the disadvantages and introducing some additional advantages.

The primary object of the invention is to coat the devices used in the medical areas by means of a device in order to avoid transmission of possible viral and communicable diseases between individuals.

An object of the invention is to allow for coating all sorts of devices required to be used in any dental/medical and sterile environment irrespective of their shapes, sizes, functions and brands by means of the device according to the invention. Making the protective cover of latex and a similar flexible material facilitates coating the devices having various forms.

Another object of the invention is to allow for performing the coating process by means of the device according to the invention without requiring manual manipulation.

Another object of the invention is to allow for removing the protective cover which has been contaminated, from the medical device by means of the device according to the invention without requiring manual manipulation.

A similar object of the invention is to shorten the cleaning stage by eliminating or reducing the contaminated surfaces of the medical devices thanks to the protective cover.

The invention aims to avoid creating the hole manually which is required for drill connections after the coating process thanks to the fact that the protective cover is open at both ends.

In order to achieve the abovementioned objects, a coating device has been developed which allows for coating the surfaces of the devices ad tools that are in direct/indirect contact with the patient in medical and dental areas. Said coating device comprises most basically at least one coating section where the coating process is carried out; an inlet opening which enables the device to be coated to enter into the coating device; at least one sealing ring which provides sealing in the coating section, is located on said inlet opening and has a geometry compatible with the coating section; a power source which provides energy for the coating device; a pump which is activated by the energy it receives from said power source; a valve which controls the entry-exit of a substance while providing negative pressure to the coating section; a connecting end which enables the coating section to be opened to said pump and valve; a connector which connects said pump, valve and connecting end; a protective cover which is placed into the coating section and has a flexible structure; and a carrier ring which fixes the protective cover onto the coating device.

In order to achieve the objects of the invention, a coating method has been developed for coating the surfaces of the devices and tools that are in direct/indirect contact with the patient in medical and dental areas by means of said coating device. This method comprises the process steps of locating the protective cover connected to the carrier ring onto the sealing ring on the coating device; generating negative pressure inside the coating section by activating the pump thanks to the energy received from the power source; inflating and enlarging of the protective cover placed inside the coating section (1) with the effect of the negative pressure; placing the medical/dental device to be coated into the protective cover which has been inflated and enlarged and taken the form of the coating section through the inlet opening; and winding the medical/dental device with the protective cover by releasing the negative pressure as a result of stopping the power source.

In order to achieve the objects of the invention, a method for removing the protective cover from the surfaces of the devices and tools which are in direct/indirect contact with the patient in medical and dental areas and has been coated by means of the coating device has been developed. This method comprises the process steps of locating the coated dental/medical device into the coating section through the inlet opening; generating negative pressure inside the coating section by activating the pump by the energy received from the power source; inflating and enlarging of the protective cover on the coated medical/dental device placed inside the coating section with the effect of the negative pressure; removing the protective cover which has been inflated and enlarged and taken the form of the coating section from the medical/dental device.

The structural and characteristic features and all the advantages of the invention will be understood more clearly thanks to the figures given below and the detailed description composed with reference to these figures; and therefore, the evaluation needs to be done by taking into consideration these figures and the detailed description.

### FIGURES TO FACILITATE UNDERSTANDING OF THE INVENTION

FIG. 1a represents a lateral perspective view of the coating device according to the invention.

FIG. 1b represents another lateral perspective view of the coating device according to the invention.

FIG. 1c represents a cross-sectional view of the coating device according to the invention.

FIG. 2a represents a disassembled alternative embodiment of the protective cover to be used for coating the medical/dental devices comprising drill or connection apparatus.

FIG. 2b represents an alternative view of the components located on the protective cover to be used for coating the medical/dental devices comprising drill or connection apparatus.

FIG. 2c represents an alternative embodiment of the protective cover to be used for coating the medical/dental devices comprising drill or connection apparatus.

FIG. 3a represents a disassembled view of the protective cover with one end thereof closed which is an alternative embodiment.

FIG. 3b represents a view of the protective cover with one end thereof closed which is an alternative embodiment.

FIG. 4a represents a disassembled view of the protective cover and the supplementary cover with one end thereof closed which is an alternative embodiment.

FIG. 4b represents a view of the protective cover and the supplementary cover with one end thereof closed which is an alternative embodiment.

FIG. 5a represents an alternative embodiment wherein the coating device of the invention is used to remove the protective cover from the coated medical/dental devices.

FIG. 5b represents a top view of the barrier used in an alternative embodiment wherein it is used to remove the protective cover from the coated medical/dental devices.

FIG. 5c represents a bottom view of the barrier used in an alternative embodiment wherein it is used to remove the protective cover from the coated medical/dental devices.

FIG. 6a represents a disassembled view of the adjustable ring.

FIG. 6b represents a view of the adjustable ring in an open position.

FIG. 6c represents a view of the adjustable ring in a closed position.

FIG. 7 represents a view of the adjustable ring placed on the glove in a closed position.

FIG. 8 represents a view of the adjustable ring placed on the glove in an open position.

The drawings do not need to be scaled necessarily and the details that are not necessary for the understanding of the present invention may have been ignored. Apart from this, the elements that are at least substantially identical or that have at least substantially identical functions are shown with the same numbers.

### DESCRIPTION OF PART REFERENCES

A. Coating device
1. Coating section
2. Inlet opening
3. Sealing ring
4. Coating section connection piece
5. Pump
6. Valve
7. Sensor
8. Connecting end
9. Connector
10. Valve hose
11. Button
12. Carrier ring
13. Gripping element
14. Fixed connection member
15. Mobile connection member
16. Protective cover
17. Open end
18. Stopper end
19. Stopper
20. Opening
21. Disc
22. Plug
23. Handle
24. Direction indicator
25. Supplementary cover
26. Supplementary cover ring
27. Supplementary cover open end
29. Closed end
30. Supplementary cover inlet opening
32. Barrier
33. Cap
34. Cap connection section
35. Power source
36. Coating section window
37. Pipe
38. Parabolic apparatus
39. Gripping section
40. Narrow section
41. Sphere
42. Glove
43. Wrist part
44. Adjustable ring

### DETAILED DESCRIPTION OF THE INVENTION

In this detailed description, preferred embodiments of the protective cover (16) coating device (A) according to the invention are described only for a better understanding of the invention without any limiting effect.

The invention is related to a protective cover (16) coating device (A) that is used to coat the devices such as medical/dental devices or a tattoo gun which need to be hygienic and carry a risk of transmitting diseases from person to person. Furthermore, the coating device (A) can also be used to wear the gloves which can be considered as a protective cover especially in a surgical environment without using hands. Said coating device (A) can also be used to coat the probes of ultrasound devices (biplanar, intra-rectal, vaginal, convex, and linear probes), endoscopic cameras, and laparoscopic cameras.

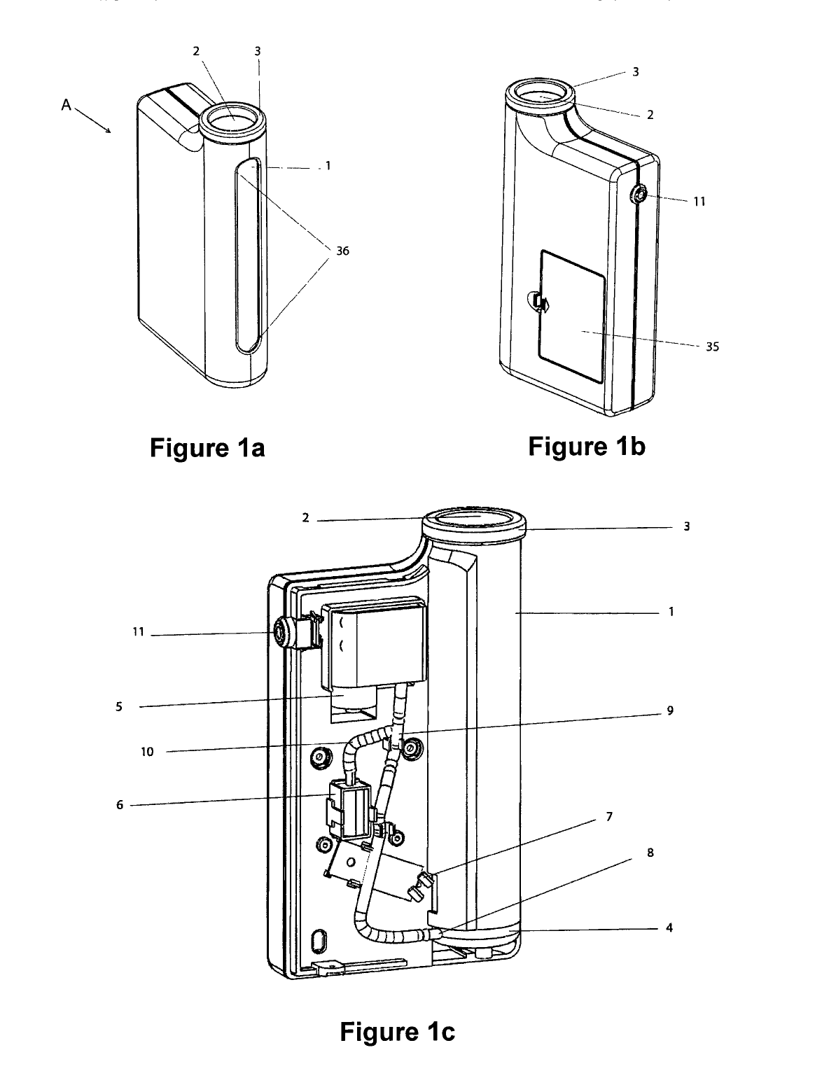

Perspective views from both sides of the coating device (A) according to the invention are given in FIGS. 1a and 1b. On the other hand, a cross-sectional view of the coating device (A) is given in FIG. 1c wherein internal parts thereof can be seen. As seen in the figure, the coating device (A) comprises at least one coating section (1) where the coating process will be carried out. Said coating section (1) is configured such that one side thereof will be open and the other side thereof will be closed. A sealing ring (3) which encircles the inlet opening (2) on the coating section (1) and provides sealing of the negative pressure generated inside the coating section is provided. Said sealing ring (3) is made of silicon material. The coating section (1) and the sealing ring (3) encircling the coating section (1) can be cylindrical or can have a different geometric form. The coating section (1) can be tetragonal prism, rectangular prism, triangular prism, elliptical, conical or reverse conical in shape or can be a combination of several geometric forms. Moreover, sizes of the coating section (1) can be changed depending on the size and shape of the device to be coated or more than one coating section (1) can be provided in one coating device (A). In conclusion, the inner diameter of the coating section (1) and the sealing rings (3) should be larger enough to incorporate several devices and tools. A coating section connection piece (4) is provided on the closed end of the coating section (1). Said connection piece (4) is connected with the pump (5) and the valve (6).

At least one pump (5) and valve (6) are contained for the negative pressure to be generated in the coating section (1). Said pump (5) discharges the air inside the coating area (1) and enables the protective cover (16) placed on the sealing ring (3) to expand inwards and inflate. Besides, said pump (5) is a vacuum or liquid pump. The valve (6), on the other hand, is responsible for entry and exit of the substance (air or liquid) used in the process that is carried out to generate negative pressure. Said valve (6) is a solenoid liquid or solenoid air valve.

In addition, the coating device (A) of the invention comprises a power source (35) to be activated. In the invention, the preferred power source is a cell battery. However; if preferred, the coating device (A) can also be activated by utilizing an external absorbing power without incorporating any electronic component and pump (5). To that end, a vacuum effect from the outside that will be connected to the connecting end (8) of the coating section (1) will be enough. Devices capable of vacuuming such as a surgical aspirator available in dental offices can be used to create a vacuum effect. The coating device (A) can be portable or it can be used as one-piece on the dental chair.

Additionally, the coating device (A) comprises some components which are employed for connecting the coating section (1), pump (5), and valve (6) to each other and allow for carrying the substance (air or liquid) that generates the negative pressure. A connecting end (8) which connects the pump (5) to the coating section (1) is provided. Besides, a connector (9) which connects the pump (5), valve (6) and connecting end (8) is contained. Said connector (9) is a T-connector. In addition, a valve hose (10) which connects said valve (6) to the connector (9) is included.

Additionally, a sensor (7) is preferably provided on the coating device (A). Said sensor (7) is situated towards the closed end of the coating section (1) and checks if the protective cover (16) reaches the base or not. When the sensor (7) detects that the protective cover (16) has reached the base, it stops the pump (5). Said sensor (7) is an infrared proximity sensor. The sensor (7) which is used to check the termination of the negative pressure can be inside or outside the coating section (1). A mechanical switch placed to the base or lateral surface inside the coating section (1) can also provide the same function instead of the sensor (7).

Besides, a coating section window (36) is preferably provided on the coating device (A). It is a section that is formed to observe the protective cover (16) inside the coating section (1).

A button (11) is provided on the coating device (A). Said button (11) allows for starting and ending the vacuuming by checking the pump (5), valve (6) and sensor (7).

The component which will be used in the coating process inside the coating device (A) is a protective cover (16). The protective cover (16) is made of an elastic material such as latex and nitrile. Raw material of nitrile covers is acrylonitrile butadiene which is a synthetic copolymer; and as it does not contain latex, it can be used as an alternative for those who are allergic to latex.

It is important to design the inner diameter of the protective cover (16) smaller than that of the dental and medical devices to be coated in order to cover the surfaces of the devices more tightly and to avoid any debris and liquid entry between the surfaces of the coated device. Therefore, diameter of the protective cover (16) should be 5-6 mm at the most.

The protective cover (16) placed on the sealing ring (3) encloses the open inlet opening (2) of the coating section (1) hermetically. The protective cover (16) is placed on the sealing ring (3) by means of a carrier ring (12). The carrier ring (12) is connected to the open end (17) of the protective cover (16). Said ring can preferably comprise a gripping element (13). The gripping element (13) allows for retaining the carrier ring (12) and removing thereof from the protective cover (16) after the coating process. Said carrier ring (12) can be made of a rigid material or of a more flexible material such as silicon and rubber.

The gripping element (13) is connected onto the carrier ring (12) such that it will make a 90-degree angle by means of a mobile connection member (15) and a fixed connection member (14). Since the gripping element (13) is connected to the carrier ring (12) by means of a mobile connection member (15), it can be moved for 180° in the reverse direction of the center of the carrier ring (12). Thanks to this movement, the protective cover (16) on the flat surface of the gripping element (13) can be pushed outside the carrier ring (12) and the carrier ring (12) can be removed easily after the coating process.

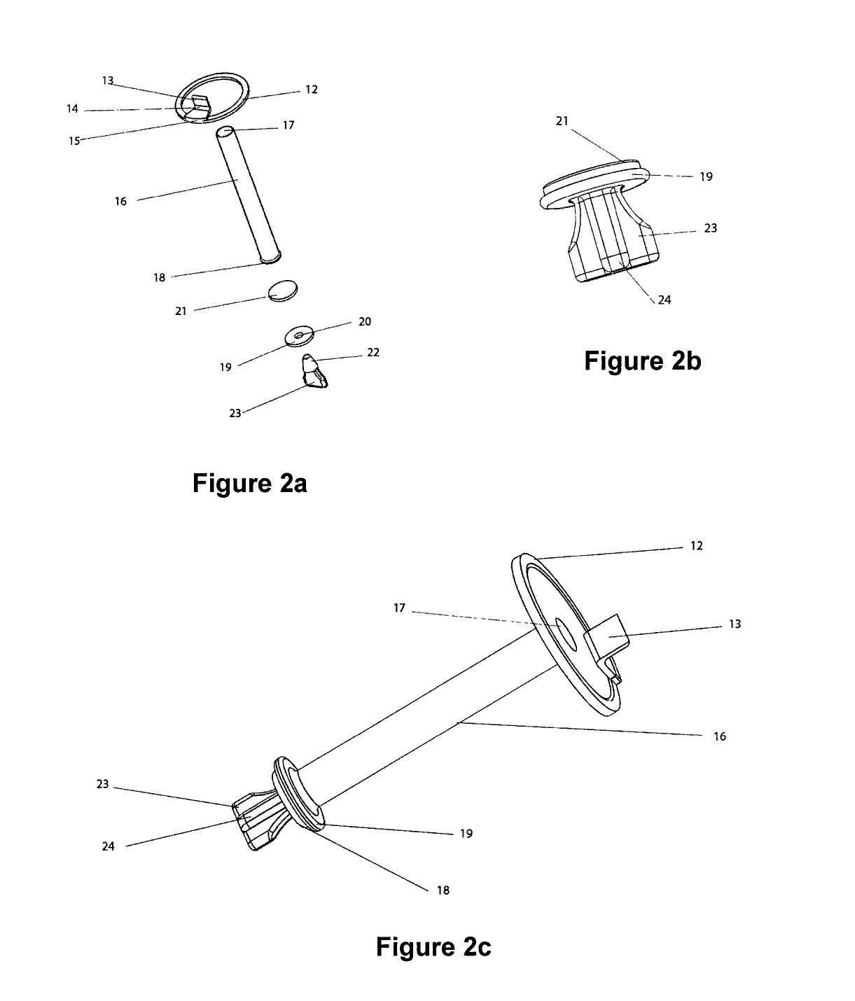

A preferred embodiment of the protective cover (16) is illustrated in FIG. 2a. In the figure, the protective cover (16) is chosen such that both ends thereof will be open. Thus, it is ensured that the device is coated such that there will be an open surface just as large as the drill or apparatus inlet opening.

In said embodiment, a gripping element (13) and a carrier member (12) are provided on the open end (17) of the protective cover (16). Said carrier ring (12) comprises a gripping element (13). A stopper end (18) is provided on the other end of the protective cover (16). The stopper (19) is provided which is connected to the stopper end (18) providing hermetical sealing and has a diameter larger than that of the stopper end (18).

An opening (20) is created at the center of the stopper (19) for drill or apparatus connection of the dental/medical devices. A complete closed structure is obtained by inserting a plug (22) into said opening (20) with a diameter that is compatible with that of the opening, wherein said plug has a handle (23) thereon.

Besides, a sponge disc (21) soaked into disinfectant is provided on the surface of the stopper (19) which remains inside the protective cover (16). Said disc (21) is integrated with the stopper (19). The sponge disc (21) soaked into disinfectant supports disinfection by contacting directly with the drill or apparatus connection surface of the device after the coating process. In addition, a direction indicator (24) which indicates the direction of placing the protective cover (16) into the coating device (A) is provided on the plug (22) with a handle (23).

FIG. 2b illustrates a preferred embodiment of the components which are connected to said protective cover (16). The stopper (19) can be produced and used as integrated with the plug (22) and the handle (23) without an opening (20) thereon. In such case, the stopper (19) is completely removed from the stopper end (18) of the protective cover (16) by means of the handle (23) after the coating process. Said disc (21) is also provided in this preferred embodiment in order to provide disinfecting property.

FIG. 2c illustrates an alternative embodiment wherein the characteristics mentioned in FIGS. 2a and 2b are employed together. In addition to said characteristics, a section which is created by curling the protective cover (16) over itself at the stopper end (18) in order to prevent the stopper (19) from being separated from the protective cover (16). Besides, the open end (17) of the protective cover (16) inserted into the carrier ring (12) is provided on the flat surface that is formed by making a 90-degree angle between gripping element (13) and the carrier ring (12).

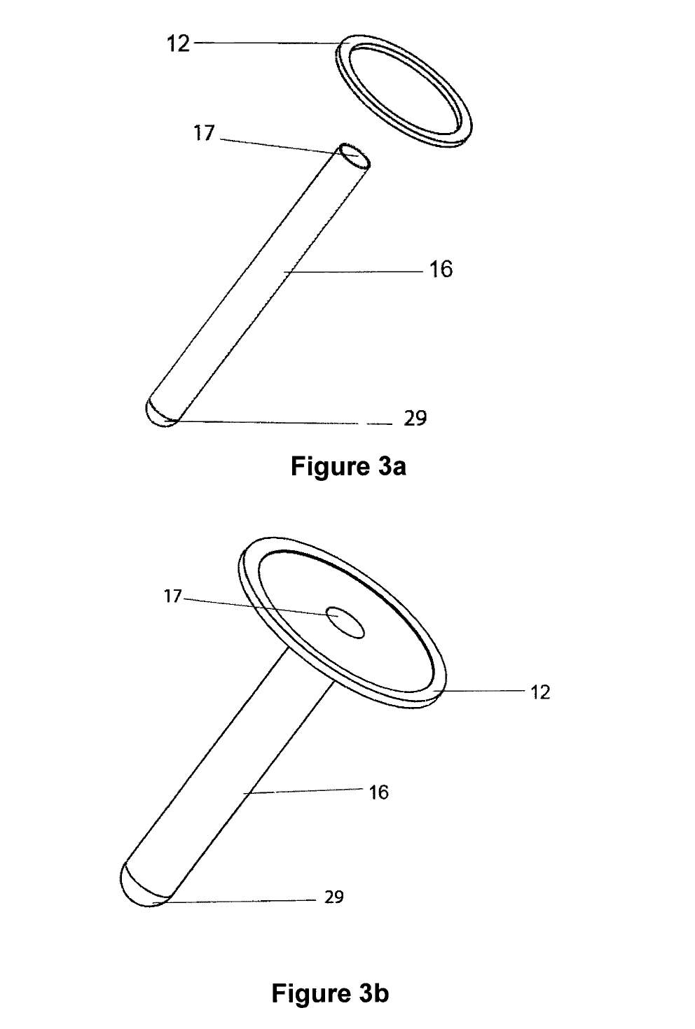

FIG. 3a illustrates another alternative embodiment of the protective cover (16). Said protective cover (16) is designed such that only one end thereof is open. Thus, it is used to coat devices which does not contain drill or apparatus connection (light curing device, intra-oral camera, digital x-ray sensor, reflector arm, etc.). Therefore, said protective cover (16) has a closed end (29). As seen in FIG. 3b, the open end (17) of the protective cover (16) with a closed end (29) is inserted into the carrier ring (12). As mentioned before, the carrier ring (12) can also comprise a gripping element (13) if preferred (not shown in the figures).

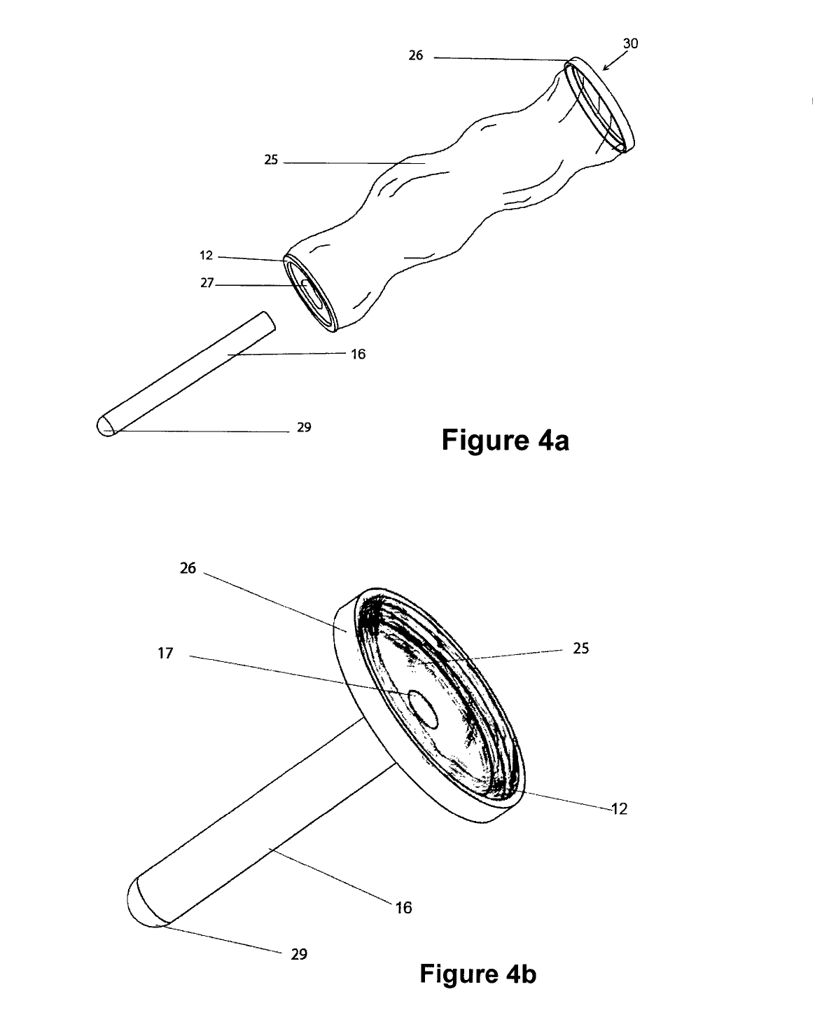

FIG. 4a illustrates another alternative embodiment of the protective cover (16). When some dental/medical devices are considered, the inner diameter of the coating section (1) and the sealing ring (2) restricts the devices and tools to be coated. A supplementary cover (25) is provided to coat the dental/medical devices which are larger than the inner diameter. The supplementary cover (25) is connected with the carrier ring (12). Said supplementary cover (25) has a structure with both ends thereof being open. The supplementary cover open end (27) is provided in the section where said supplementary cover is connected with the carrier ring. A supplementary cover inlet end (30) of the supplementary cover (25) is provided at the other end of the supplementary cover (25) and the supplementary cover ring (26) is integrated with the supplementary cover (25) at this end.

The supplementary cover open end (27) can be combined and used with the protective cover (16). In this manner, the parts of dental/medical devices which do not fit into the coating device (A) can be coated by means of the supplementary cover (25).

The use of protective cover (16) together with the supplementary cover (25) is illustrated in FIG. 4b. The carrier ring (12) which is compatible with the geometry and diameter of the supplementary cover ring (26) that is made of a rigid or semi-rigid material is placed such that it will be inside the supplementary cover (25) on the side of the supplementary cover open end (27). A combination of the protective cover (16) and the supplementary cover (25) is obtained by inserting a protective cover (16) into the carrier ring (12). Then, the supplementary cover (25) is brought together inside the supplementary cover ring (26) and the supplementary cover ring (26) is inserted onto the carrier ring (12).

For the medical/dental devices such as light curing devices and wireless endodontic micromotors hand-held parts of which are large, the parts which enter into the coating section (1) are coated with the protective cover (16) while the parts which are larger than the inner diameter of the coating section (1) and the sealing ring (3) are coated with the supplementary cover (25).

In said supplementary cover (25)-protective cover (16) combination, the protective cover (16) with one end thereof closed as in FIG. 3b can be employed if there is not any drill and apparatus connection, and the embodiments as in FIGS. 2a and 2c can be employed if there is a drill and apparatus connection.

### Operating Principle:

Before operating the coating device (A), the protective cover (16) (with one end or both ends thereof open or in combination with the supplementary cover (25)) should be selected based on the shape of the dental/medical device to be coated. Afterwards, the protective cover (16) is connected with the carrier ring (12). Then, the carrier ring (12) connected with the protective cover (16) is inserted onto the sealing ring (3) on the coating section (1). Thus, the inlet opening (2) of the coating section (1) is hermetically closed.

When the on-off button (11) is pressed, the solenoid air valve (6) hinders the air intake and the vacuum pump (5) starts to suck air from the coating section (1) to which it is connected.

As soon as the activated pump (5) starts to discharge air from the coating section (1), a negative pressure occurs inside the coating section (1). Thanks to the generated negative pressure, the protective cover (16) enlarges and starts to inflate inside the coating section (1).

When the protective cover (16) which moves forward inside the coating section (1) reaches the bottom of the coating section (1), it triggers the infrared proximity sensor (7). The activated proximity sensor (7) stops the vacuum pump (5) and warns the user audibly and visually. This prevents the vacuum pump (5) from operating for a long time and provides energy saving. Afterwards, the device or tool to be coated is placed into the latex protective cover (16) that has been enlarged through the inlet opening (2) of the coating section (1).

After this process step, the button (11) is pressed again and the solenoid air valve (6) and the vacuum pump (5) are completely deactivated; and as a result, air is let into the coating section (1). The protective cover (16) which has been inflated due to the pressure change inside the coating section (1) starts to shrink and encircle the device placed therein tightly. The device inside the coating section (1) is removed from the device as covered with the protective cover (16).

The carrier ring (12) with or without a gripping element (13) can be removed from the protective cover (16) or left thereon optionally and depending on the area of use of the coated device. If the carrier ring (12) has a gripping element (13), the carrier ring (12) can be easily removed by pulling from the gripping element (13). Thanks to the movement of the mobile connection member (15) of the gripping element (13) that is connected to the carrier ring (12), it can be moved for 180° in the reverse direction of the center of the carrier ring (12). Thanks to this movement, the protective cover (16) on the fixed connection member (14) of the gripping element can be pushed outside the carrier ring (12) and the carrier ring (12) can be easily removed from the protective cover (16). If the coated device has a drill or apparatus connection, the plug (22) with a handle (23) at the stopper end (18) of the protective cover (16) is removed from the protective cover (16). Besides, the opening (20) of the stopper (19) is adjusted by being centered such that it will correspond to the drill or apparatus connection hole of the coated device. In this manner, a surface only as large as the drill or apparatus inlet space of the coated device will be left open and the stopper (19) with an opening (20) will be located on the coated device and under the protective cover (16).

Removal of the Device Coated with the Protective Cover (16) after Use:

In order to remove the protective cover (16) seen in FIGS. 2a and 2c which is used especially for the devices with a drill and apparatus hole, the open end (17) of the protective cover (16) on the coated device is forwarded by being manually rolled towards the stopper end (18) of the protective cover (16) and removed from the coated device.

In order to remove the protective covers (16) as in FIGS. 3b and 4b, the coated material needs to be placed into the coating section (1) through the inlet opening (2) of the coating device (A) and the device needs to be activated. The protective cover (16) on the coated material enlarges and inflates inside the coating section (1) with the effect of negative pressure. Thus, the protective cover (16) is separated from the coated device. The user finishes the process by taking the device that is released in the inflated protective cover (16) outside the coating section (1).

The use of two separate devices for the process of coating and removing the protective cover (16) is important in the control of cross-infection. For this reason, it is highly important to make available two separate devices and not to confuse one with the other in practice.

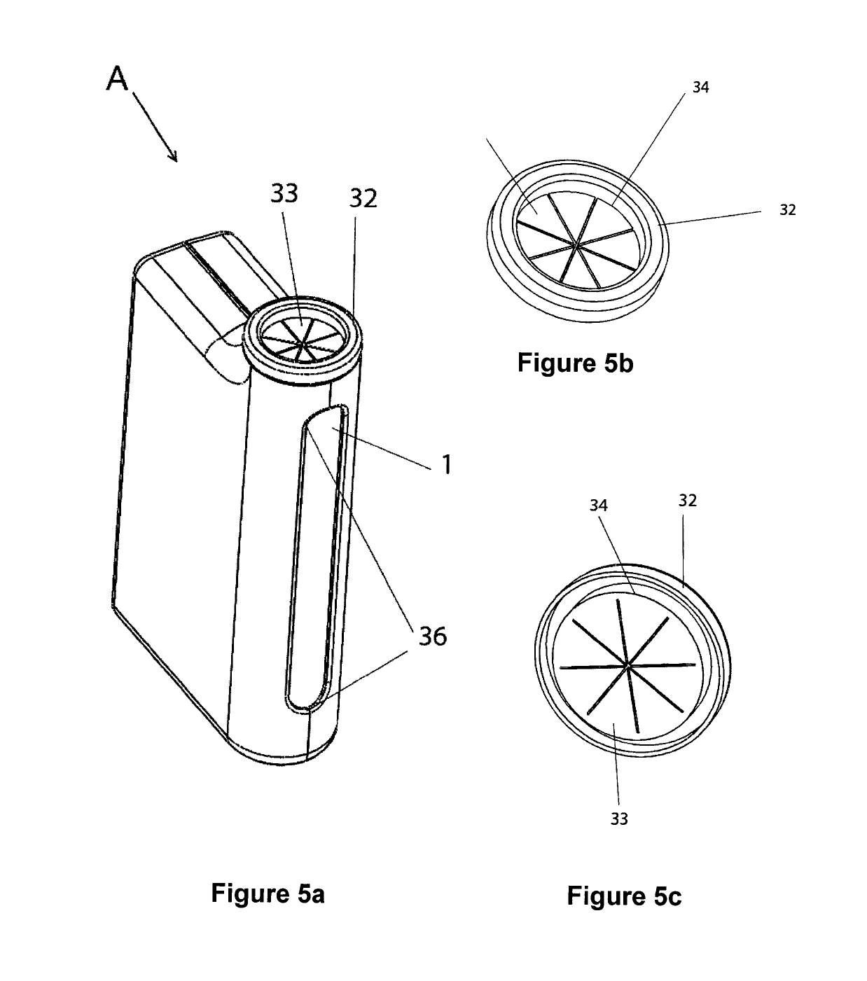

FIG. 5a illustrates an alternative embodiment of the device (A) to be used in the process of removing the protective cover (16). A barrier (32) hindering the inlet is provided in the inlet opening (2) of the coating section (1) instead of the sealing ring (3).

FIGS. 5b and 5c illustrate top and bottom views of said barrier (32) respectively. Said barrier (32) is triangular in shape and made of silicon and it consists of caps (33). The cap connection area (34) which is the large side of these triangular caps (33) is combined with the inner surface of the barrier (32). Said caps (33) have an elastic and mobile structure so as to let only the coated medical/dental device enter into the coating section (1). Thus, the use of the device (A) with a barrier (32) attached thereto in the process of coating protective cover (16) by the user is avoided both physically and visually.

Use of the Coating Device (A) for the Process of Wearing a Glove without Contact:

It is quite important to check the medical gloves whether they are pierced or not before and after use. Required measures should be taken according to the area and risk of use of the person using the glove which is detected as pierced after use.

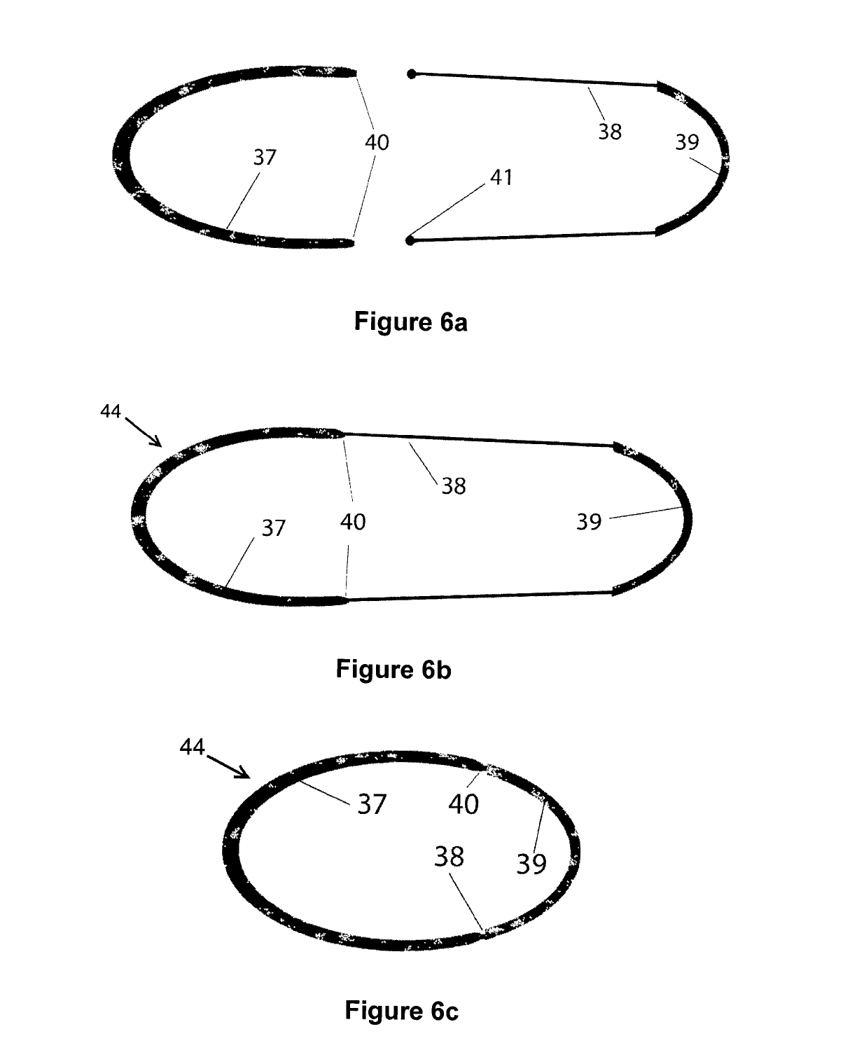

The coating device (A) of the invention can be used to wear the medical gloves. It can be used to wear and take off the gloves (42) by designing the gloves (42), which have the inlet opening (2) of the coating device (A) and sealing ring (3) designs as shown in FIG. 6b, with a diameter and geometry compatible with the open position of the adjustable ring (44).

The adjustable ring (44) which will be attached to the wrist part (43) of the medical gloves (42) is shown in FIGS. 6a, 6b and 6c. The adjustable ring (44) consists of a pipe (37) made of parabolic polyurethane material suitable for the wrist anatomy of a human being and a parabolic apparatus (38) moving inside said pipe (37). The parabolic apparatus (38) can be made of plastic or metal material.

The adjustable ring (44) is shown in two positions as open and closed in FIGS. 6b and 6c respectively. The diameter of the parabolic apparatus (38) is smaller than the inner diameter of the pipe (37). Thus, the parabolic apparatus (38) can easily move inside the pipe (37). In order for the adjustable ring (44) to stay in the open position inside the medical glove (42), spheres (41) are provided at both end of the parabolic apparatus (38). The diameter of these spheres (41) is smaller than the inner diameter of the pipe (37) and larger than the narrow parts (40) at the ends of the pipe (37). As such, when the parabolic apparatus (38) easily moves inside the pipe (37) and the spheres (41) at the end of the apparatus (38) reach the narrow part (40) at the end of the hose, the spheres (41) get stuck in this section and enable the adjustable ring (44) to stay in the open position.

There is a gripping section (39) on the parabolic apparatus (38) of the adjustable ring (44). As seen in FIG. 6c, said gripping section (39) ensures integrity of the pipe (37) in the closed position of the adjustable ring (38). Besides, it makes it easy to hold the gripping section (39) while the adjustable ring (44) is being brought to the open position. The gripping section (39) of the adjustable ring (44) is adjusted to the wrist part (43) of the medical glove (42) such that it will be on the side of the thumb of the glove (42). Thus, the adjustable ring (44) can be easily opened and closed manually before and after use.

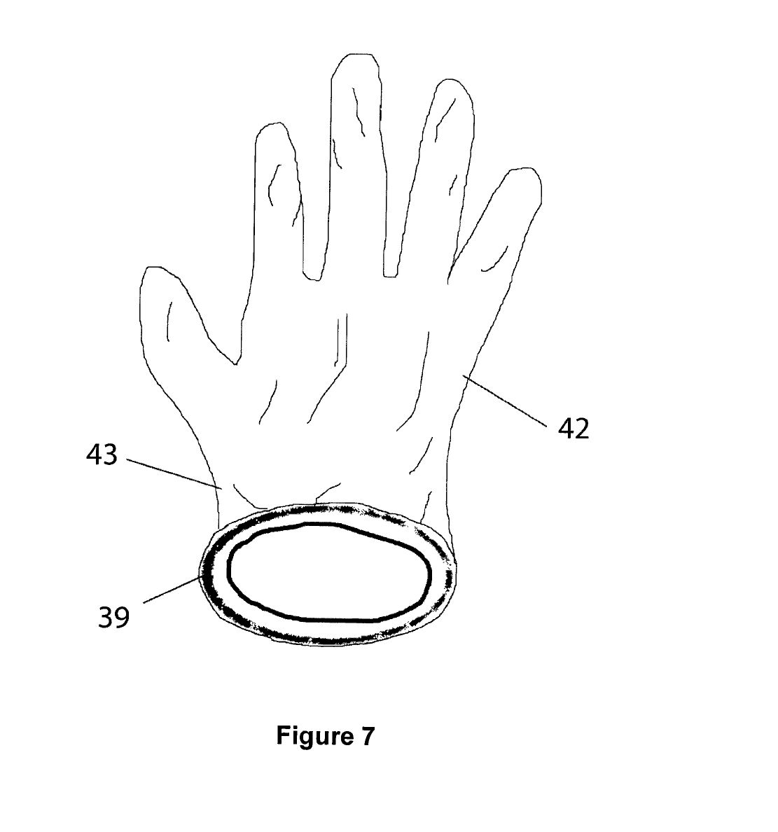

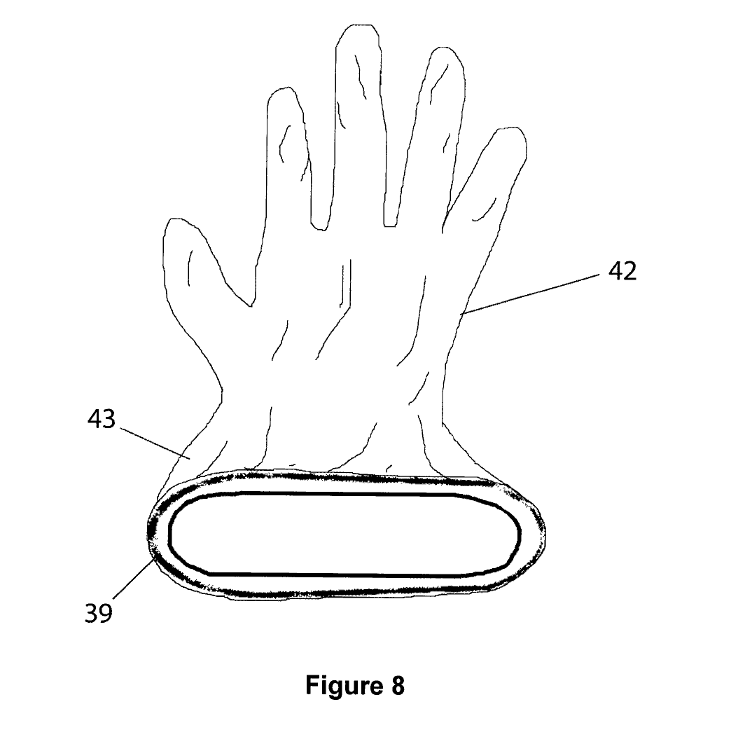

The closed position of adjustable ring (44) that is attached to the wrist part (43) of the medical glove (42) is seen in FIG. 7 while the open position thereof is seen in FIG. 8.

The use of different coating devices (A) while wearing and taking off the medical gloves (42) is important in terms of sterilization.

1. A coating device allowing for coating the surfaces of the devices and tools that are in direct/indirect contact with the patient in medical and dental areas, characterized in comprising:
at least one coating section where the coating process is carried out,
an inlet opening which enables the device to be coated to enter into the coating device,
at least one sealing ring which provides sealing in the coating section, is located on said inlet opening and has a geometry compatible with the coating section,
a power source which provides energy for the coating device,
a pump which is activated thanks to the energy it receives from said power source,
a valve which controls the entry-exit of a substance while providing negative pressure to the coating section,
a connecting end which enables the coating section to be opened to said pump and valve,
a connector which connects said pump, valve and connecting end,
a protective cover which is placed into the coating section and has a flexible structure, and
a carrier ring which fixes the protective cover onto the coating device.

2. The coating device as in claim 1, characterized in comprising an infrared proximity sensor or a mechanical switch which controls the enlargement of the protective cover with the effect of the negative pressure.

3. The coating device as in claim 1, characterized in comprising a valve hose which connects said valve to the connector.

4. The coating device as in claim 1, characterized in comprising a button which turns on/off said coating device.

5. The coating device as in claim 1, characterized in that said carrier ring comprises a gripping element.

6. The coating device as in claim 4, characterized in that said gripping element comprises a fixed connection member and a mobile connection member.

7. The coating device as in claim 1, characterized in that said protective cover is latex or nitrile.

8. The coating device as in claim 1, characterized in that said protective cover comprises an open end and a stopper end in order to be used in medical/dental devices with drill or connection apparatus.

9. The coating device as in claim 8, characterized in that a stopper connected to the stopper end of said protective cover is provided.

10. The coating device as in claim 9, characterized in comprising an opening on said stopper.

11. The coating device as in claim 9, characterized in comprising a sponge disc which is soaked into disinfectant and integrated with said stopper.

12. The coating device as in claim 9, characterized in comprising a plug and a handle connected to said stopper.

13. The coating device as in claim 12, characterized in comprising a direction indicator connected to said plug and handle.

14. The coating device as in claim 1, characterized in that said protective cover comprises an open end and a closed end in order to be used in medical/dental devices without drill or connection apparatus.

15. The coating device as in claim 14, characterized in comprising a supplementary cover which is connected to said protective cover by means of the carrier ring.

16. The coating device as in claim 15, characterized in that said supplementary cover comprises a supplementary cover open end and a supplementary cover inlet end.

17. The coating device as in claim 16, characterized in that said supplementary cover comprises a supplementary cover ring at the supplementary cover inlet end.

18. The coating device as in claim 1, characterized in comprising a barrier when said coating device is used to remove the coated protective covers.

19. The coating device as in claim 18, characterized in that said barrier comprises caps.

20. The coating device as in claim 1, characterized in that said protective cover is a glove.

21. The coating device as in claim 20, characterized in that said glove comprises an adjustable ring thereon.

22. The coating device as in claim 21, characterized in comprising a pipe, a parabolic apparatus, a narrow section and a sphere on said adjustable ring.

23. The coating device as in claim 1, characterized in comprising a coating section window through which the coating section can be seen.

24. A method for coating the surfaces of the devices and tools that are in direct/indirect contact with the patient in medical and dental areas by means of said coating device as in claim 1, characterized in comprising the following process steps;
locating the protective cover connected to the carrier ring onto the sealing ring on the coating device;
generating negative pressure inside the coating section by activating the pump thanks to the energy received from the power source;
inflating and enlarging of the protective cover placed inside the coating section with the effect of the negative pressure;
placing the medical/dental device to be coated into the protective cover which has been inflated and enlarged and taken the form of the coating section through the inlet opening; and
winding the medical/dental device with the protective cover by releasing the negative pressure as a result of stopping the power source.

25. A method for removing the protective cover from the surfaces of the devices and tools which are in direct/indirect contact with the patient in medical and dental areas and has been coated by means of said coating device as in claim 1, characterized in comprising the following process steps;
locating the coated dental/medical device into the coating section through the inlet opening;
generating negative pressure inside the coating section by activating the pump thanks to the energy received from the power source;
inflating and enlarging of the protective cover on the coated medical/dental device placed inside the coating section with the effect of the negative pressure; and
removing the protective cover which has been inflated and enlarged and taken the form of the coating section from the medical/dental device.

26. The coating/removing method as in claim 24, characterized in that the process step of pressing the button is realized with the energy received from the power source.

27. The coating/removing method as in claim 24, characterized in comprising the process step of detecting the inflation and enlargement of the protective cover by means of the sensor on the coating device and of stopping the power source by activating a valve.
